(19) United States
(12) Patent Application Publication
(10) Pub. No.: US 2003/0049335 A1
(43) Pub. Date: Mar. 13, 2003

(54) Edible compositions comprising freeze-dried flavoring agents

(76) Inventors: Roger E. Stier, Clifton, NJ (US); John Zanone, Montville Township, NJ (US)

Correspondence Address: DAVINCI-ONLINE(TM) BY THE PERSON OF Y. F, RAGOS, 37 POSIDONOS ST VOULA, ATHENS, GR16673, GR

(21) Appl. No.: 10/285,245

(22) Filed: Oct. 31, 2002

Related U.S. Application Data
(62) Division of application No. 09/871,334, filed on May 31, 2001.

(51) Int. Cl. A61K 35/78

(52) U.S. Cl. 424/725; 424/732; 424/736; 424/747; 424/742

(57) ABSTRACT

The invention pertains to edible compositions comprising flavoring agents and active ingredients of such agents comprising freeze-dried fruits, herbs, vegetables, spices or extracts. The invention also concerns pharmaceutical compositions comprising the freeze-dried fruits, herbs, vegetables, spices or extracts, including tablets, coated tablets, suspensions and liquid solutions having active pharmaceutical agents for treating upper gastrointestinal tract distress, and methods for treating upper gastrointestinal tract distress in humans.

### BACKGROUND OF THE INVENTION

1. Field of the Invention

The invention pertains to edible compositions that comprise freeze-dried flavoring agents from natural fruits, herbs, vegetables, spices, extracts or combinations thereof. The invention also concerns pharmaceutical compositions comprising the freeze-dried flavoring agents, including tablets, suspensions and liquid solutions having active ingredients for treating upper gastrointestinal tract distress, and methods for treating upper gastrointestinal tract distress in humans. Other examples of edible compositions comprising the freeze-dried flavoring agents are confectionary products, including chewing gum, hard and soft candies and the like.

2. The Prior Art

Flavoring agents are used in edible goods to provide particular flavor notes to edible compositions. Recent trends indicate that natural flavoring agents are preferred by consumers over artificial or non-natural agents. Thus, the art is constantly evolving with respect to freeze-dried fruit flavorings and edible compositions comprising freeze-dried fruit flavoring, particularly in the pharmaceutical and confectionary industry, as well as other industries associated with the manufacture of edible goods. For example, WO 99/44436 describes a chewing gum comprising a dried natural flavoring agent, which may be fruit flavoring, applied to the coating of the chewing gum.

In pharmaceutical compositions, such as antacids, useful for treating upper gastrointestinal tract distress, such as heartburn, indigestion, stomachache and the like, flavorings, such as fruit flavors, are generally employed as excipients to provide an appealing and pleasing flavor to the composition. Customarily, artificial flavorings are used for pharmaceutical compositions such as antacids because of cost and processing convenience. There is a growing consumer demand, however, for use of natural and non-modified ingredients, such as natural flavorings. Also, the use of natural flavorings alleviates certain regulatory requirements and approvals in the manufacture of edible goods and compositions, such as pharmaceuticals.

It was an object of the invention to develop new comestible goods and edible compositions, like pharmaceutical compositions and confectionary products, comprising natural flavoring agents and/or active components of such agents in solid freeze-dried form.

It was a further object of the invention that the natural flavoring agents and/or active components of such agents contain natural fruits, herbs, vegetables, spices or extracts, and the like, so that inclusion of such flavoring agents supports a labeling claim of natural, contains real fruit or the like.

It was yet a further object of the invention to develop pharmaceutical compositions, such as antacids, comprising natural freeze-dried flavoring agents.

These, and other, objects have been achieved by the flavoring agents and comestible goods and edible compositions described herein. The freeze-dried flavoring agents or active components of such agents comprise natural fruits, herbs, vegetables, spices or extracts. When the flavoring agent or active component is exposed to moisture, such as saliva, the freeze-dried component becomes re-hydrated and provides a burst of flavoring. Because the flavoring agent or active component is natural, products comprising the flavoring agents will support commercial labeling that the product is natural, contains real fruit and the like.

In the present Specification, all parts and percentages are by weight/weight unless otherwise specified. The term “by weight of the flavoring agent components” as used herein means the weight percentage based on the total weight of all of the components of the flavoring agent, including the active component, moisture and other components. The term “by weight of the pharmaceutical composition” as used herein means the weight percentage based on the total weight of all components of the pharmaceutical composition.

### SUMMARY OF THE INVENTION

The invention pertains to edible compositions comprising natural, freshly harvested flavoring agents or active components of such agents in freeze-dried powdered form. The flavoring agents can be used in a number of products, including in the preferred embodiment of the invention, pharmaceutical compositions, particularly those for treating upper gastrointestinal tract distress. The flavoring agents may also be used in other comestible goods, e.g. edible compositions, including confectionary goods, such as candies. The flavoring agents are re-hydrated when exposed to moisture in the human mouth which provides a burst of flavor.

The freeze-dried flavoring agents are made from freshly harvested fruits, herbs, vegetables, spices or extracts thereof which are frozen and then subjected to a refrigerated vacuum system. The freeze-dried fruit, herb, vegetable, spice or extract may be in any size or shape having intact cell structure, and can be used in the form of a ground powder of the intact cell structure.

The freeze-dried flavoring agents can be used alone, or in conjunction with other flavoring agents, such as spray-dried flavorings, in edible compositions. The freeze-dried flavoring agent provides for burst release of flavor when the composition is consumed. The invention pertains to edible compositions comprising freeze-dried flavoring agents or the active components of such agents from natural or freshly harvested fruits, herbs, vegetables, spices, extracts or combinations thereof.

In a preferred embodiment of the invention the flavoring agent is incorporated into a pharmaceutical composition, like those in the form of a tablet for treating gastrointestinal tract distress in humans. The pharmaceutical composition may be coated with edible coating materials, like edible resins such as a shellac or glaze. In addition to the freeze-dried flavoring agent, the pharmaceutical compositions may also comprise natural or artificial spray dried flavorings as part of the flavoring agent or separate from the agent.

### DETAILED DESCRIPTION OF THE INVENTION

The invention pertains to edible compositions comprising freeze-dried flavoring agents and active components of such agents comprising freeze-dried fruits, herbs, vegetables, spices, extracts or combinations thereof and the like. Examples of edible compositions are confectionary products, such as candy, gum, syrups and the like; beverages, such as juices, teas, carbonated beverages and the like; cakes; breads and other comestible goods and pharmaceutical compositions. In a most preferred embodiment of the invention, the flavoring agents are used in antacid formulations, particularly antacid tablets, and the antacid tablets comprising the flavoring agents may be coated with a shellac or glaze or other edible resin or coating material. Because the freeze-dried flavoring agent is made from natural ingredients, such as freshly harvested and frozen ingredients, the flavoring agent supports a labeling claim of natural, contains real fruit or the like. When exposed to moisture, such as moisture in the human mouth, the flavoring agent re-hydrates and provides a burst of flavor.

The flavoring agent or active component may comprise natural and naturally identical fruits, herbs, vegetables, spices, extracts and the like, and combinations thereof, which also includes plant extracts. Examples of fruits useful as an active component of the flavoring agent are those selected from the group consisting of orange, lemon, mango, pineapple, lime, strawberry, cherry, black currant, red currant, blueberry, raspberry, wild berry, cranberry, apple, pear, banana, prune, plums, coconut, grapefruit, mandarin, grape, gooseberry, lingonberry, chokeberry, chokecherry, mixtures thereof and the like. Examples of extracts that can be used in the flavoring agent are those selected from the group consisting of peppermint, periwinkle, eucalyptus, spearmint, anethol, menthol, anise, mixtures thereof and the like. Other flavoring agents or active components are plant extracts selected from the group consisting of liquorice, coffee, tea, mixtures thereof and the like; herbs selected from the group consisting of sage, thyme, bergamont, balm, valerian, camomile, lavender, aloe vera, mixtures thereof and the like; and spices selected from the group consisting of pepper, cinnamon, capsicum, paprika, tarragon, fennel, mustard, dill, caraway, parsley, tomato, mixtures thereof and the like. Combinations of the above flavoring agents or active components may also be used. It is understood that the foregoing are merely descriptive as any fruit, vegetable, herb, spice or extract can be used so long as it may be freeze-dried.

Freeze-dried flavoring agents and active components have the advantage of providing more intense flavor and increased stability because many of the flavor notes of the taste remain present in the more or less intact cells of the fruits, herbs, vegetables, spices or extracts. That is, even when used in the form of a powder, the fruit, herb, vegetable, spice or extract will comprise particles having an intact cell structure which, we have discovered, enables a flavor burst compared to other natural or artificial flavoring. The moisture content of the flavoring agent is also relevant to the stability and must be up to about 75% by weight of the flavoring agent components, more preferably about 10% to about 60% by weight of the flavoring agent components, most preferably between about 15% and about 30% by weight of the flavoring agent components. The flavoring agent is generally used in the form of a powder, however, freeze-dried slices or pieces and combinations thereof with or without powder may also be used. Generally, the freeze-dried fruit, herb, vegetable, spice, or extract, will have a particle size of up to about 3 millimeters, preferably having a particle size range from about 3 microns to about 2 millimeters. Preferred ground powders have a particle size such that about 100% of the ground particles pass through a U.S.#40 mesh screen.

Flavorings may also be used in addition to or in conjunction with the freeze-dried fruit flavoring agents or active components thereof. Spray-dried fruit or other flavoring may be used, and the spray-dried fruit may have the same or different flavor from the freeze-dried flavoring agent or active component. Spray-dried fruit, however, will not provide the benefits of the freeze-dried fruit and may not support desired label claims. Spray-dried flavorings may be used as an excipient in pharmaceutical composition embodiments, as an ingredient in other embodiments and as a component of the flavoring agent where the flavoring agent comprises an active component of freeze-dried fruit, herb, vegetable, spice or extract.

The freeze-dried fruit flavoring agents are used as an ingredient in edible compositions for flavoring purposes. Because the freeze-dried fruit flavorings comprise natural, freshly harvested fruits, herbs, vegetables, spices, extracts thereof or combinations thereof, the edible composition will be characterized by a stronger and more enhanced flavor release or profile, and can support a labeling claim of natural, contains real fruit or the like. Embodiments of the invention pertain to edible compositions comprising the freeze-dried flavoring agents described herein from natural, or freshly harvested, fruits, herbs, vegetables, spices, extracts or combinations thereof.

The flavoring agents, or active agents thereof, are made by a freeze-drying process using a refrigerated vacuum system. Fresh harvested fruits, herbs, vegetables, spices, or extracts thereof, are quick frozen and placed in a refrigerated vacuum system so that ice in the fruit, herb, vegetable, spice or extract is converted directly into water vapor. This process does not disturb cell structure and the freeze-dried product retains the color, shape and nutritional value of the freshly harvested fruit, herb, vegetable, spice, or extract thereof. The refrigerated vacuum system also provides for rapid dehydration which, in part, provides beneficial characteristics to the freeze-dried substrate, such as flavor burst upon rehydration during consumption. The freeze-dried product may be of any shape or size, but preferably the freeze-dried product is ground into a powder from intact freeze-dried cell structure. Freeze-dried fruits, herbs, vegetables, spices or extracts of several varieties may be used in the flavoring agent, or one variety of such may be used.

When used in pharmaceutical compositions, particularly those for treating upper gastrointestinal tract distress, such as heartburn, indigestion, stomachache and the like, the flavoring agents are excipients which may be combined with active pharmaceutical agents and other excipients and then compressed into tablets or used in liquid form, such as solutions, suspensions or emulsions and the like. Because the flavoring agent comprises a natural active component, any pharmaceutical composition comprising the flavoring agent may have commercial labeling that the pharmaceutical composition is natural, contains real fruit or the like. In addition to providing flavor burst and supporting labeling claims, the freeze-dried fruit flavoring agent or active ingredient of such agent masks chalky taste generally encountered with antacid formulations, such as tablets, solutions or suspensions.

Active pharmaceutical agents for treating upper gastrointestinal tract distress are those materials which are safe and effective when administered orally for treating disorders of the upper gastrointestinal tract (typically the stomach and/or esophagus) which result in symptoms of upper gastrointestinal tract distress. Such active pharmaceutical agents include antacid agents and acid secretion prevention agents (e.g., H₂ receptor-blocking antisecretory agents). Antacid agents include, for example, aluminum carbonate, aluminum hydroxide, aluminum phosphate, aluminum hydroxy-carbonate, dihydroxy aluminum sodium carbonate, aluminum magnesium glycinate, dihydroxy aluminum amino acetate, dihydroxy aluminum aminoacetic acid, calcium carbonate, calcium phosphate, aluminum magnesium hydrated sulfates, magnesium aluminate, magnesium alumina silicates, magnesium carbonate, magnesium glycinate, magnesium hydroxide, magnesium oxide, magnesium trisilicate, sucralfate, and mixtures thereof. Examples of acid secretion prevention agents include cimetidine, ranitidine, famotidine, omeprazole, and mixtures thereof. Other useful active pharmaceutical agents include bismuth-containing agents such as, for example, bismuth subsalicylate, bismuth aluminate, bismuth citrate, bismuth subcitrate, bismuth nitrate, bismuth subcarbonate, bismuth subgalate, and mixtures thereof. A particularly preferred bismuth salt is bismuth subsalicylate.

Preferred antacid agents are aluminum hydroxide, magnesium hydroxide, dihydroxy aluminum sodium carbonate, calcium carbonate, and mixtures thereof. Most preferred is calcium carbonate.

The pharmaceutical compositions comprise a safe and effective amount of at least one active pharmaceutical agent, useful for treating upper gastrointestinal tract distress. Typically the active pharmaceutical agent(s) are from about 1% to about 99%, preferably from about 30% to about 40%, by weight of the pharmaceutical composition. The pharmaceutical compositions also comprise the flavoring agents which are generally present in the pharmaceutical compositions in an amount of about 0.01% to about 2%, preferably about 0.5% to about 1.5%, by weight of the pharmaceutical composition.

In addition, excipients other than the flavoring agents may optionally be included in the pharmaceutical compositions. The term “excipient(s)”, as used herein, means, in addition to the flavoring agent, one or more compatible solid or liquid fillers, diluents or encapsulating substances which are suitable for oral administration to a human and encompasses all of the ingredients of the pharmaceutical compositions except the active pharmaceutical agent. The term “compatible”, as used herein, means that the components of the compositions of the invention are capable of being commingled with the active pharmaceutical agent, and with each other, in a manner such that there is no interaction which would substantially reduce the pharmaceutical efficacy of the compositions under ordinary use situations. Excipients must, of course, be of sufficiently high purity and sufficiently low toxicity to render them suitable for administration to the human being.

Some examples of substances which can serve as excipients are sugars such as lactose, glucose and sucrose; starches such as cornstarch and potato starch; cellulose and its derivatives such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobroma; polyols such as propylene glycol, glycerine, sorbitol, mannitol, and polyethylene glycol; agar; and alginic acid; as well as other non-toxic compatible substances used in pharmaceutical formulations. Flavorings, such as spray dried flavors may also be used as excipients in the pharmaceutical compositions. Wetting agents and lubricants such as sodium lauryl sulfate, as well as coloring agents, sweetening agents (including nonnutritive sweeteners such as aspartame and saccharine), tableting agents, stabilizers, antioxidants, and preservatives, can also be present. Other compatible pharmaceutical additives and actives which are not active pharmaceutical agents useful for treating upper gastrointestinal tract distress (e.g., NSAI drugs; pain killers; muscle relaxants) may be included in the compositions of the invention. Materials having cooling properties, cooling agents, may optionally be included as the excipients, such as menthol, compounds comprising N-substituted p-menthane-3-carboxamides (such as N-ethyl p-methane-3-carboxamide), 3,1-methoxy propane 1,2-diol and the like, or combinations thereof.

The choice of excipients to be used in conjunction with the active pharmaceutical agent is basically determined by the dose form for the pharmaceutical compositions. The preferred dosage forms are tablets, especially chewable tablets, capsules and the like, comprising a safe and effective amount of the active pharmaceutical agent(s). Dosage forms may also include liquid solutions, liquid suspensions and the like. Excipients suitable for the preparation of dosage forms for oral administration are well-known in the art. Their selection will depend on secondary considerations like taste, cost, shelf stability, and can be made without difficulty by a person skilled in the art.

The excipients employed in the ingestible compositions are used at concentrations sufficient to provide a practical size to dosage relationship. Typically, excipients comprise from about 1% to about 99%, preferably from about 85% to about 99%, by weight of the pharmaceutical composition.

The pharmaceutical composition in tablet form may be coated, which reduces instability of the freeze-dried flavoring agent or active component of the flavoring agent. The coating material, which is generally an edible resin applied to the tablet in the form of a film, comprises about 0.1% to about 2.0%, preferably about 0.5% to about 1.5%, by weight of the pharmaceutical composition on a dry weight basis. The freeze-dried flavoring agent or active component thereof is preferably contained within the tablet and not the coating, however the coating can, if desired, comprise the freeze-dried flavoring agent.

Coating stabilizes certain freeze-dried flavoring agents or active components in the tablet formulation which become unstable when exposed to atmospheric conditions and/or light particularly when stored for periods of time at elevated temperature. The instability alters the flavoring characteristics. For example, freeze-dried raspberry flavoring agents in tablet form pharmaceutical compositions may experience a flavoring change from a sweet berry taste to a berry taste having off-notes when stored at elevated temperatures. In addition, if the tablet comprises spray dried flavorings, these flavorings would likewise be stabilized by the coating.

The coating may be in the form of an edible resin, such as an aqueous or alcoholic resin. Shellac is a form of resin that may be used in the coating, such as CertiSeal FC 300 A and CertiSeal A100 shellac available from Mantrose-Haeuser Company, Westport, Conn., USA (“Mantrose Haeuser”) which is prepared from a resinous secretion of Laccifer Lacca that is dissolved in warm soda water, bleached with hypochlorite and, optionally, dewaxed. Alcoholic glazes from shellac may also be used to advantage, such as 4# Refined Pharmaceutical Glaze (NF in a specially denatured alcohol formula 45/200) containing about 35% solids content available from Mantrose-Haeuser. Preferably, the solids content of the coating material will be from about 1% to about 45%.

The coating may impart bitter tones to the overall flavor of the tablet. Accordingly, sweeteners, such as saccharin, sucralose, stevia or combinations thereof, and in an amount of about 0.1% to about 1%, by weight of the pharmaceutical composition, may be incorporated into the coating material or resin. Additionally the coating material or resin may comprise natural flavoring agents, artificial flavoring agents or combinations thereof, such as those selected from the group consisting of menthol, peppermint oil and the like or combinations thereof. The coating material may also comprise other additives, fillers, coloring agents, cooling agents and the like.

The tablet comprising the freeze-dried flavoring agent, other excipients and active pharmaceutical agent can be coated by a spray gun process to obtain a film coating on the tablet. A solution or suspension comprising the aqueous or alcoholic resin, optional sweetener, and other additives, fillers and coloring is formed by standard mixing or blending techniques. The tablets are placed into a tumbling bed or fluidized bed of multi-particulates in which the coating is applied by use of a spray gun. After the liquid coating is applied, the tablets are allowed to dry so that gellation of the coating and adhesion of the film to the tablet occurs. The parameters of the coating operation, as would be understood by one skilled in the art, will depend on the operation and coating type. Some of the conditions to be considered in developing coating parameters are concentration of polymer or solids in solution, atomizing air pressure, liquid flow rate, gun to substrate distance, spray gun design and shape, liquid nozzle diameter and atomizing air velocity.

The pharmaceutical compositions comprising the flavoring agents described herein can be employed in methods for treating upper gastrointestinal tract distress in humans. The method comprises orally administering to a human a safe and effective amount of at least one active pharmaceutical agent useful for treating upper gastrointestinal tract distress. The preferred mode of administration for this method is through an ingestible composition, most preferably through an ingestible tablet.

### EXAMPLE 1

Sugar based antacid tablets comprising freeze-dried raspberry flavor and calcium carbonate active pharmaceutical agent were prepared. The tablets were prepared by combining 5% by weight of the pharmaceutical composition ground raspberry flavoring from a Van Druen Farms, Momence, Ill., 38% by weight of the pharmaceutical composition calcium carbonate (USP, BC)from Whittaker, Clark & Daniels, Inc., South Plainfield, N.J. USA, 52% by weight of the pharmaceutical composition granular sugar commercially available from sources such as DOMINO® sugar from Amstar Sugar Corporation, New York, N.Y., USA and other fillers and additives in dry form in a Cole-Parmer laboratory mixer from Cole-Parmer Instrument Company, Vernon Hills, Ill., USA and mixing for 30 minutes at speed 50 revolutions per minute until the components were completely mixed. After mixing, the components were pressed into tablets weighing about 1.0 gram each by using a Cherry-Burrell rotary tablet press.

The tablets were subjected to a stability test through storage at both ambient temperature and 50° C. for one week. After storage at these conditions for one week the tablets were observed to be stable in that no color changes or structural abnormalities were noted.

The tablets were subjected to a tasting test using five panelists. Each panelist separately sampled one tablet stored at ambient temperature for one week and one tablet stored at 50° C. for one week. The testing protocol required each panelist to place a tablet in the mouth, chew twenty times and swallow The panelists were asked to report flavor perception during consumption. The tablets stored at ambient temperature were reported by the panelists as having an acceptable and pleasing flavor. The panelists reported that the flavoring of the tablets stored at 50° C. for one week had wheat flavor tones to the raspberry flavor, compared to the tablets stored at ambient temperature.

### EXAMPLE 2

Tablets formulated in accordance with Example 1 were coated with Refined Pharmaceutical Glaze 4# in NF specially denatured alcohol formula 45/200 from Mantrose-Haeuser using a spray gun on a tray. Tablets were coated with sufficient coating to have 0.7% glaze by weight of the pharmaceutical composition on a dry weight basis and 1.5% glaze by weight of the pharmaceutical composition on a dry weight basis. After coating, the tablets were dried first by air blowing for 1 hour at ambient conditions (temperature of 25° C., relative humidity at 34%), then using a forced air oven at 38° C. for 30 minutes. The coated tablets were then permitted to further dry at ambient conditions overnight.

The coated tablets having a 0.7% glaze and a 1.5% glaze were subjected to a stability test through storage at both ambient temperature and 50° C. for one week. After storage at these conditions for one week, both the coated tablets with 0.7% glaze and the coated tablets with 1.5% glaze were observed to be stable in that no color changes or structural abnormalities were noted.

The coated tablets were subjected to a tasting test using five panelist. Each panelist separately sampled one each of the coated tablets having a 0.7% glaze and a 1.5% glaze stored at ambient temperature and stored at 50° C. for one week. The testing protocol for required each panelist to place a coated tablet in the mouth, chew twenty times and swallow. This was repeated for each of the total of four tablets sampled by the panelists. The panelists were asked to report flavor perception during consumption. The coated tablets stored at ambient temperature and at 50° C. having 0.7% glaze and 1.5% glaze were reported by the panelists as having an acceptable and pleasing flavor.

1. An edible composition comprising a freeze-dried flavoring agent or active component having an average particle size of up to about 3 millimeters and a moisture content of up to about 75% by weight of the flavoring agent components, wherein the flavoring agent or active component comprises freeze-dried freshly harvested fruit, herb, vegetable, spice, extract or combinations thereof.

2. The edible composition of claim 1 wherein the moisture content is about 10% to about 60% by weight of the flavoring agent components.

3. The edible composition of claim 1 wherein the fruit is selected from the group consisting of orange, lemon, mango, pineapple, lime, strawberry, cherry, black currant, red currant, blueberry, raspberry, wild berry, cranberry, apple, pear, banana, prune, plums, coconut, grapefruit, mandarin, grape, gooseberry, lingonberry, chokeberry, chokecherry and mixtures thereof.

4. The edible composition of claim 1 wherein the extract is selected from the group consisting of peppermint, periwinkle, eucalyptus, spearmint, anethol, menthol, anise, liquorice, coffee, tea and mixtures thereof.

5. The edible composition of claim 1 wherein the herb is selected from the group consisting of sage, thyme, bergamont, balm, valerian, camomile, lavender, aloe vera and mixtures thereof.

6. The edible composition of claim 1 wherein the spice is selected from the group consisting of pepper, cinnamon, capsicum, paprika, tarragon, fennel, mustard, dill, caraway, parsley, tomato and mixtures thereof.

7. The edible composition of claim 1 wherein the freeze-dried freshly harvested fruit, herb, vegetable, spice or extract is ground.

8. The edible composition of claim 7 wherein the ground fruit, herb, vegetable, spice or extract has a particle size such that about 100% of the ground particles pass through a U.S. #40 mesh screen.

9. A pharmaceutical composition comprising from about 1% to about 99%, by weight of the pharmaceutical composition, of at least one active pharmaceutical agent selected from the group consisting of antacid agents, acid secretion prevention agents, bismuth containing agents and mixtures thereof and from about 1% to about 99%, by weight of the pharmaceutical composition, excipients comprising at least one flavoring agent wherein the flavoring agent comprises an active agent selected from the group consisting of freeze-dried fruit, herb, vegetable, spice, extract and combinations thereof.

10. The pharmaceutical composition of claim 9 wherein the fruit is selected from the group consisting of orange, lemon, mango, pineapple, lime, strawberry, cherry, black currant, red currant, blueberry, raspberry, wild berry, cranberry, apple, pear, banana, prune, plums, coconut, grapefruit, mandarin, grape, gooseberry, lingonberry, chokeberry, chokecherry and mixtures thereof.

11. The pharmaceutical composition of claim 9 wherein the extract is selected from the group consisting of peppermint, periwinkle, eucalyptus, spearmint, anethol, menthol, anise, liquorice, coffee, tea and mixtures thereof.

12. The pharmaceutical composition of claim 9 wherein the moisture content of the flavoring agent is up to about 75% by weight of the flavoring agent components.

13. The pharmaceutical composition of claim 9 wherein the moisture content of the flavoring agent is about 10% to about 60% by weight of the flavoring agent components.

14. The pharmaceutical composition of claim 9 wherein the flavoring agent has an average particle size of up to about 3 millimeters.

15. The pharmaceutical composition of claim 9 wherein the freeze-dried fruit, herb, vegetable, spice or extract is ground.

16. The pharmaceutical composition of claim 9 in the form of a tablet.

17. The pharmaceutical composition of claim 16 wherein the tablet is coated with an edible resin.

18. The pharmaceutical composition of claim 17 wherein the edible resin is an aqueous or alcoholic resin.

19. The pharmaceutical composition of claim 17 wherein the edible resin comprises shellac.

20. The pharmaceutical composition of claim 17 wherein the resin comprises saccharin, sucralose, stevia or combinations thereof.

21. The pharmaceutical composition of claim 17 wherein the resin comprises natural flavoring agents, artificial flavoring agents or combinations thereof.

22. The pharmaceutical composition of claim 21 wherein the flavoring agents are selected from the group consisting of menthol, peppermint oil and combinations thereof.

23. The pharmaceutical composition of claim 17 comprising about 0.1% to about 2% edible resin by weight of the pharmaceutical composition on a dry weight basis.

24. The pharmaceutical composition of claim 9 further comprising spray-dried flavorings.

25. The pharmaceutical composition of claim 9 wherein the antacid agents are selected from the group consisting of aluminum carbonate, aluminum hydroxide, aluminum phosphate, aluminum hydroxy-carbonate, dihydroxy aluminum sodium carbonate, aluminum magnesium glycinate, dihydroxy aluminum amino acetate, dihydroxy aluminum aminoacetic acid, calcium carbonate, calcium phosphate, aluminum magnesium hydrated sulfates, magnesium aluminate, magnesium alumina silicates, magnesium carbonate, magnesium glycinate, magnesium hydroxide, magnesium oxide, magnesium trisilicate, sucralfate, and mixtures thereof.

26. The pharmaceutical composition of claim 9 wherein the acid secretion prevention agents are selected from the group consisting of cimetidine, ranitidine, famotidine, omeprazole, and mixtures thereof.

27. The pharmaceutical composition of claim 9 wherein the bismuth containing agents are selected from the group consisting of bismuth subsalicylate, bismuth aluminate, bismuth citrate, bismuth subcitrate, bismuth nitrate, bismuth subcarbonate, bismuth subgalate, and mixtures thereof.

28. The pharmaceutical composition of claim 9 further comprising cooling agents.

29. The pharmaceutical composition of claim 28 wherein the cooling agents are selected from the group consisting of menthol, compounds comprising N-substituted p-menthane-3-carboxamides, 3,1-methoxy propane 1,2-diol and combinations thereof.

30. A method for treating upper gastrointestinal tract distress in humans comprising orally administering through an ingestible composition a safe and effective amount of at least one active pharmaceutical agent and a freeze-dried flavoring agent comprising an active agent selected from the group consisting of freeze-dried fruit, herb, vegetable, spice, extract, and combinations thereof.

31. The method of claim 30 wherein the ingestible composition is in the form of a tablet.
